(19) United States
(12) Patent Application Publication
(10) Pub. No.: US 2004/0053425 A1
(43) Pub. Date: Mar. 18, 2004

(54) Quantitative measurement of proteins using genetically-engineeredglucose oxidase fusion molecules

(75) Inventors: Richard E. Link, Pikesville, MD (US); Ronald A. Morton, Richmond, TX (US); Brian Miles, Houston, TX (US); Michael Simon, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine

(21) Appl. No.: 10/430,509

(22) Filed: May 6, 2003

Related U.S. Application Data
(63) Continuation-in-part of application No. 10/419,438, filed on Apr. 21, 2003.
(60) Provisional application No. 60/374,215, filed on Apr. 19, 2002.

(51) Int. Cl. G01N 33/553

(52) U.S. Cl. 436/526

(57) ABSTRACT

Custom-engineered glucose oxidase fusion proteins, prepared by recombinant DNA techniques, are employed in a chip-based amperometric immunosensor. This on-chip assay provides quantitative measurement of analyte concentration in any fluid, including all body fluids. The system is designed to facilitate ease in swapping of molecular recognition components and can be rapidly adapted to measure the concentration of any peptide or protein for which a monoclonal antibody is available.

### CROSS-REFERENCE TO RELATED APPLICATIONS

This application is a continuation-in-part of U.S. application Ser. No. 10/419,438 which was filed on Apr. 21, 2003, and is hereby incorporated by reference in its entirety, which claims the benefit of U.S. Provisional Application No. 60/374,215, which was filed Apr. 19, 2002, and is hereby incorporated by reference in its entirety.

### TECHNICAL FIELD

The invention relates to biosensors which are capable of detecting the presence of a biomarker.

### BACKGROUND OF THE INVENTION

Immunoassay techniques are based on the ability of antibodies to form complexes with the corresponding antigens or haptens. This property of highly specific molecular recognition of antigens by antibodies leads to high selectivity of assays based on immune principles. The high affinity of antigen-antibody interactions results in great sensitivity of immunoassay methods. The use of a label or indicator to verify that an antigen/antibody interaction has occurred is the basis for immunoassay methods.

Immunoassay techniques have been used mainly in clinical analyses and medical diagnostics. However, immunoassay applications in other areas such as environmental control, food quality control, etc. are expanding. Certain limitations in assaying techniques due to existing procedures have limited somewhat the expansion into such other areas.

During the last few years a significant number of publications have dealt with non-conventional (alternative) immunoassay techniques designed to expand the accuracy or applicability of immunoassays. In most cases the development of alternative immunoassay techniques aims at improvements in performance of conventional immunoanalysis. Often such improvement attempts are directed to decreasing analysis times, increasing assay sensitivity, and simplifying and automating assay procedures.

For example, the utilization of enzymes able to catalyze electrochemical reactions by direct (mediatorless) mechanism (bioelectrocatalysis) would allow for the detection of immuno-interactions in real time. Such applications of bioelectrocatalysis in the development of immunosensors are based on the self-assembling or displacement of molecule/label complexes or “molecular transducers” on the surface of an electrode that has been modified by immunospecies that bind the complex. Ordinarily these immunospecies would be complimentary to the immunoconjugate which includes the electrocatalytically active enzyme-label.

Immunosensors utilize antibodies as binding agents. Antibodies are protein molecules that bind with specific foreign entities, called antigens, that can be associated with disease states. Antibodies attach to antigens and either remove the antigens from a host and/or trigger an immune response. Antibodies are quite specific in their interactions and, unlike enzymes, they are capable of recognizing and selectively binding to very large bodies such as single cells. Thus, antibody-based biosensors allow for the identification of certain pathogens such as dangerous bacterial strains.

There are several classes of sensors that make use of applied electrical signals for determination of analyte presence. “Amperometric” sensors make use of oxidation-reduction chemistries in which electrons or electrochemically active species are generated or transferred due to analyte presence. An enzyme that interacts with an analyte may produce electrons that are delivered to an appropriate electrode; alternately an amperometric sensor may employ two or more enzyme species, one interacting with analyte, while the other actually generates electrons as a function of the action of the first enzyme (a “coupled” enzyme system). Glucose oxidase has been used frequently in amperometric biosensors for glucose quantification for diabetics. Other amperometric sensors make use of electrochemically active species whose presence alter the system applied voltage as recorded at a given sensor electrode. Not all sensing systems can be adapted for electron generation or transfer, and thus many sensing needs cannot be met by amperometric methods alone. The general amperometric method makes use of an applied voltage and effects of electrochemically active species on said voltage. An example of an amperometric sensor is described in U.S. Pat. No. 5,593,852, which describes a glucose sensor that relies on electron transfer effected by a redox enzyme and electrochemically-active enzyme cofactor species.

An additional class of electrical sensing systems includes those sensors that make use primarily of changes in an electrical response of the sensor as a function of analyte presence. Some systems pass an electric current through a given medium; if analyte is present, there is a corresponding change in exit electrical signal, and this change implies that analyte is present. In some cases, the binding agent-analyte complex causes an altered signal, while in other systems, the bound analyte itself is the source of changed electrical response. Such sensors are distinguished from amperometric devices in that they do not necessarily require the transfer of electrons to an active electrode. Sensors based on the application of an electrical signal are not universal, in that they depend on alteration of voltage or current as a function of analyte presence; not all sensing systems can meet such a requirement. An example of this class of sensors is U.S. Pat. No. 5,698,089 which describes a chemical sensor in which analyte detection is determined by charge of an applied electrical signal. Binding of analyte to chemical moieties arranged in an array alters the conductivity of the array points; unique analytes can be determined by the overall changes in conductivity of all of the array points.

In biosensor diagnostic devices, the assay substrate and detector surface are integrated into a single device. One general type of biosensor employs an electrode surface in combination with current or impedance measuring elements for detecting a change in current or impedance in response to the presence of a ligand-receptor binding event. This type of biosensor is disclosed, for example, in U.S. Pat. No. 5,567,301.

Gravimetric biosensors employ a piezoelectric crystal to generate a surface acoustic wave whose frequency, wavelength and/or resonance state are sensitive to surface mass on the crystal surface. The shift in acoustic wave properties is therefore indicative of a change in surface mass, e.g., due to a ligand-receptor binding event. U.S. Pat. Nos. 5,478,756 and 4,789,804 describe gravimetric biosensors of this type.

Biosensors based on surface plasmon resonance (SPR) effects have also been proposed, for example, in U.S. Pat. Nos. 5,485,277 and 5,492,840. These devices exploit the shift in SPR surface reflection angle that occurs with perturbations, e.g., binding events, at the SPR interface. Finally, a variety of biosensors that utilize changes in optical properties at a biosensor surface are known, e.g., U.S. Pat. No. 5,268,305.

Biosensors have a number of potential advantages over binding assay systems having separate reaction substrates and reader devices. One important advantage is the ability to manufacture small-scale, but highly reproducible, biosensor units using microchip manufacturing methods, as described, for example, in U.S. Pat. Nos. 5,200,051 and 5,212,050. Another advantage is the potentially large number of different analyte detection regions that can be integrated into a single biosensor unit, allowing sensitive detection of several analytes with a very small amount of body-fluid sample. Both of these advantages can lead to substantial cost-per-test savings.

Other advantages of this technology, most notably speed of measurement and ease of miniaturization, make it attractive for “point of service” applications. Biosensors are being developed for measurement of pollutants in water samples in the field, for continuous blood glucose sensing in an implantable artificial pancreas, and for detection of chemical warfare agents on the battlefield.

Biosensor devices can be broken down into three general classes of utilization: external diagnostic, endoscopically deployed and implantable. The most straightforward of these types is the “external diagnostic device” which analyzes fluid or tissue immediately after its removal from the body. The most successful of these devices have been amperometric biosensors that measure blood glucose. In the presence of glucose, immobilized glucose oxidase (Gox) on these chips generates hydrogen peroxide that can be detected electrochemically. This technology is sensitive, specific, inexpensive to produce and simple to operate, making it ideal for commercial handheld glucose monitors. However, the extension of this technology to measure protein analytes has been problematic. A wide variety of biosensors have been developed that couple immune recognition with either optical, piezoelectric or electrochemical detection. Although practical for the laboratory bench, these sensors have proven difficult to translate into clinical application primarily due to labile bio-recognition components and over-engineered and expensive transducer systems.

Prostate cancer is the most common solid malignancy in men and the second most common cause of male cancer-specific mortality. Over the past fifteen years, the development and implementation of testing for PSA has revolutionized the diagnosis and treatment of this important disease. Current testing methods remain both inconvenient and costly, with conservative estimates that place the projected cost of PSA testing for screening purposes alone at greater than a billion dollars a year in the United States. These characteristics impact particularly on the population of low-income patients who may be uninsured or live in underserved areas. A disposable PSA biosensor chip would form the core of an inexpensive handheld device for measuring PSA at the bedside, in the physician's office or even in the home. Ideally this device would function much in the fashion of handheld monitors for blood glucose. It would require only a few drops of blood from a fingerstick and provide reproducible quantitative results within fifteen minutes. This device could greatly facilitate mass public screening for prostate cancer by providing PSA results at the screening site and eliminating the difficult task of following up on thousands of delayed blood test results.

### BRIEF SUMMARY OF THE INVENTION

An embodiment of the present invention is a method of detecting a biological marker having a specific anti-marker antibody comprising obtaining a sample; adding the sample to a detection device, which in specific embodiments comprises reaction cell comprising catalase and glucose, a recombinant fusion protein characterized by a redox activity and an immunoreactivity against a capture antibody, and an electrode having immobilized on its surface the capture antibody and a biomolecular peroxide sensor; applying an electrical signal to the mixture; and measuring a magnitude of a current generated in the detection device, wherein the magnitude of the generated current is inversely proportional to the concentration of biological marker in the sample.

In a specific embodiment, the biological marker is a polypeptide having an epitope that binds specifically to the capture antibody. In another specific embodiment, the biological marker is a tumor marker. The tumor marker may be PSA, HK2, TGFβ, her2, CA 15-3, CA-125, Cyfra 21-1, CEA, CD151, TPA, TPS, chromogrannin A, neuron specific enolase, β-HCG, α-fetoprotein, LDH, or any tumor marker known in the art. In another specific embodiment, the tumor marker binds specifically to the capture antibody. In one embodiment of the invention, the anti-marker antibody and the capture antibody are the same. In another embodiment of the invention, the sample comprises whole blood, serum, plasma, urine, or saliva.

In a further specific embodiment of the invention, the redox activity is provided by a polypeptide of a glucose oxidase. In yet another specific embodiment of the invention, immunoreactivity is provided by a polypeptide comprising an epitope of a tumor marker. In a specific embodiment if the invention, the recombinant fusion protein is prepared in yeast. The yeast may be methyltrophic yeast. In a specific embodiment if the invention, the recombinant fusion protein is prepared by expressing a polynucleotide comprising both a glucose oxidase and the epitope, wherein the glucose oxidase and the epitope are operatively linked.

In a specific embodiment, the biomolecular peroxide sensor comprises a horseradish peroxidase. In another specific embodiment, the capture antibody is immobilized by an interaction between the biotin and avidin. In a further specific embodiment, the electrical signal comprises a voltage of about +50 mV. The measuring step comprises a potentiostat in yet another embodiment of the invention. In a specific embodiment, the potentiostat is capable of measuring a current in the range of about 50 nanoampere to about 500 nanoampere.

Also provided in the invention is a method of screening a patient for cancer comprising: obtaining a sample from the patient; forming a reaction mixture by adding the sample to a reaction cell comprising catalase and glucose, a recombinant fusion protein characterized by a redox activity and an immunoreactivity against a capture antibody, and an electrode having immobilized on its surface the capture antibody and a biomolecular peroxide sensor; applying an electrical signal to the reaction mixture; and measuring a magnitude of a current generated in the reaction mixture, wherein the magnitude of the generated current is inversely proportional to the concentration of biological marker in the sample; and determining the presence of a cancer in the patient from the concentration of the tumor marker in the sample. In another specific embodiment of the invention, the solution comprises about 1% glucose.

Another embodiment of the present invention is a kit for screening a patient comprising: a reaction cell comprising catalase and glucose; a recombinant fusion protein characterized by a redox activity and an immunoreactivity against a capture antibody; an electrode having immobilized on its surface the capture antibody and a biomolecular peroxide sensor, and a potentiostat.

An embodiment of the present invention is a disposable biosensor for screening for the presence of a biological marker in a sample comprising: a reaction cell comprising a solution of catalase and glucose; a recombinant fusion protein characterized by a redox activity and an immunoreactivity against a capture antibody; and an electrode having immobilized on its surface the capture antibody and a biomolecular peroxide sensor.

An embodiment of the invention is a handheld biological marker detection device comprising: a reaction cell comprising catalase and glucose, a recombinant fusion protein characterized by a redox activity and an immunoreactivity against a capture antibody, and an electrode having immobilized on its surface the capture antibody and a biomolecular peroxide sensor; and a potentiostat. In certain embodiments, the recombinant fusion protein comprises SEQ ID NO:2 and SEQ ID NO:1.

An embodiment of the invention is a composition comprising SEQ ID NO:1. Another embodiment of the invention is a recombinant fusion protein comprising SEQ ID NO: 1 and a glucose oxidase.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same putposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows an outline of the reaction mechanism of the biosensor chip.

FIG. 2 shows the mapping of the peptide recognition epitope for the anti-PSA antibody ER-PR8 using a synthetic peptide library. FIG. 2A shows the design of the 83 overlapping 15 mer peptides. FIG. 2B shows the results of an ELISA experiment using biotinylated ER-PR8 antibody with the peptides. Antibody binding was detected using an ABC detection kit with horseradish peroxidase (Vector Laboratories, Burlingame, Calif.) and ABTS as the chromogenic substrate. This graph shows the absorbance at 405 nm which is proportional to antibody binding peptide. Peptides 52-54 showed the highest amount of binding.

FIG. 3 shows the determination of the consensus epitope.

FIG. 4 shows mapping the consensus epitope onto the three-dimensional structure of human PSA.

FIG. 5 shows Western blotting analysis to demonstrate binding of anti-PSA antibody to ER-PR8 and not native Gox.

FIG. 6 shows PCR amplification of full length coding sequence for glucose oxidase (Penicillium amagasakiense).

FIG. 7 shows the screening of Pichia clones for Gox activity.

FIG. 8 shows Western blot analysis of conditioned media from a Pichia clone with vector control or with pPicZ-GPM6 at varying time points.

FIG. 9 shows SDS-PAGE analysis of purified fusion protein.

FIG. 10 shows quantitative dot blot analysis of PSA, and purified fusion protein.

FIG. 11 shows the purification as following Gox activity of the yeast-expressed fusion protein.

FIG. 12 shows the three electrode configuration for the PSA biosensor chip design. The electrode is screen-printed with a 2 mm carbon working electrode containing horseradish peroxidase, a 1 mm² printed Ag/AgCl reference electrode and a crescent-shaped Ag counter electrode.

FIG. 13 shows the current response of unmodified electrodes to hydrogen peroxide. Electrodes were incubated in phosphate buffer and a constant voltage of +50 mV Vs. Ag/AgCl was applied. Hydrogen peroxide was added over the concentration range of 12.5 to 150 μM. Current response was defined as the current measured from 80 to 100 seconds following initial voltage application. FIG. 13A shows raw data after addition of hydrogen peroxide. FIG. 13B shows the standard curve for current response to

FIG. 14 shows the current response of unmodified electrodes to horseradish peroxidase after the application of +50 mV Vs. Ag/AgCl. Electrodes were exposed to increasing concentrations of purified glucose oxidase from Aspergillus niger in phosphate buffer containing 1% glucose. Di/dt was calculated 15 to 60 seconds after enzyme addition, during the linear phase of current rise. FIG. 14A shows representative raw data for a single chip exposed to varying glucose oxidase concentrations. FIG. 14B indicates the standard curve for di/dt with increasing concentrations of glucose oxidase.

FIG. 15 shows the localization of glucose oxidase to either the surface microenvironment or bulk solution determines the current response of the biosensor electrodes. After application of a +50 mV vs. Ag/AgCl potentail, the current generated in response to addition of a solution containing 1% glucose and 0.5 mg/mL catalase was recorded. FIG. 15A shows raw data from a representative experiment. FIG. 15B shows the peak current as defined as the average current measured from 40 to 60 seconds after substrate addition.

FIG. 16 shows the sequence of a recombinant glucose oxidase comprising SEQ ID NO:2 with the ER-PR8 epitope, SEQ ID NO:1, embedded within the coding sequence at the carboxy-terminal end. This figure depicts SEQ ID NO: 4.

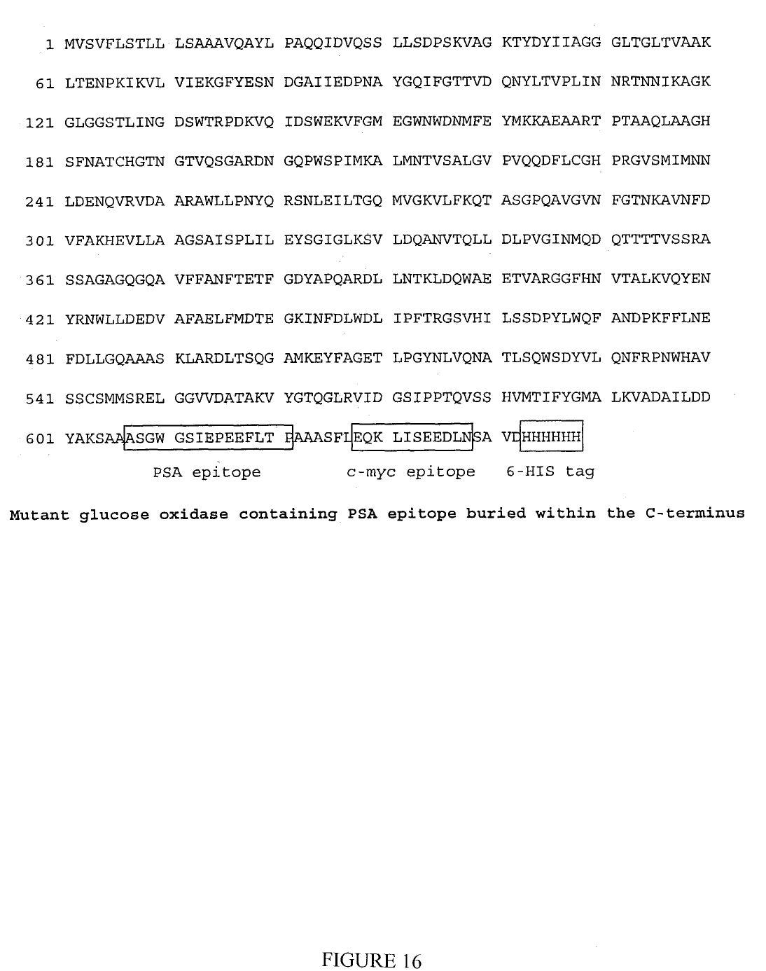

FIG. 17 shows the sequence of a recombinant glucose oxidase comprising SEQ ID NO:2 with the ER-PR8 epitope, SEQ ID NO:1, embedded within the coding sequence at the N-terminal end. This figure depicts SEQ ID NO:5.

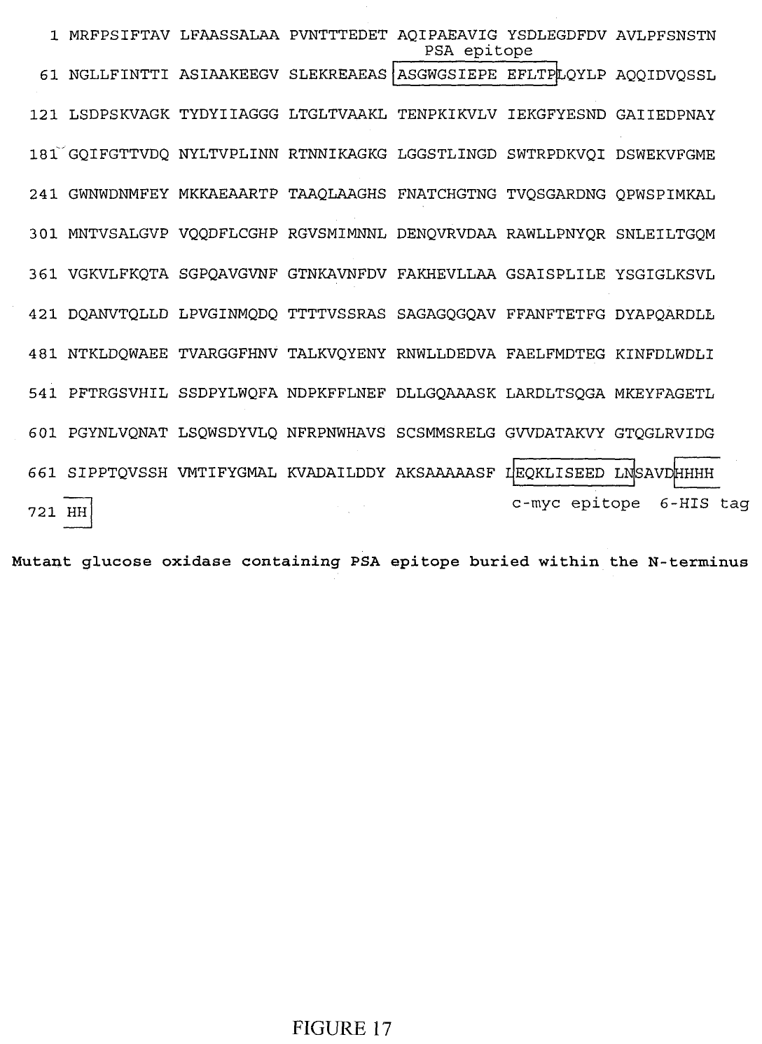

### DETAILED DESCRIPTION OF THE INVENTION

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “antibody” is intended to refer broadly to any immunologic binding agent such as IgG, IgM, IgA, IgD and IgE. An “anti-marker” antibody refers to an antibody that is specific for both an epitope contained within a biomarker of interest and also a fusion protein containing the same epitope, or antibody recognition sequence, of the biomarker, and a redox protein. As used herein, a “capture antibody” serves to bind the analyte of interest and the fusion protein to the biological peroxide sensor provided on the biosensor chip. The capture antibody is immobilized on the biosensor chip. The capture antibody may be specific for the anti-marker antibody, or may be the same as the anti-marker antibody.

As used herein, the term “biological marker” or “biomarker” refers to a substance which, when measured, may be used to assess a change or effect in a biological system. A biomarker may be used as index of the risk or progression of disease. A biomarker specifically utilized in the context of cancer, cancer diagnosis, or cancer screening, may be referred to as a “tumor marker”. Examples of known tumor markers in the art are PSA, HK2, TGFβ, her2, CA 15-3, CEA, CA-125, Cyfra 21-1, CD151, TPA, TPS, chromogrannin A, neuron specific enolase, β-HCG, α-feto protein, and LDH.

As used herein, the term “combination” may be used to refer to a mixture, a blend, or a composite. Such a combination may be dry, or in solution. One with skill in the art realizes that a combination is not limited to a particular order in which one places the components of said combination.

As used herein, the phrase “current measurement” refers to the electrical measurement by which the analyte concentration is monitored. Current measurement can be continuous or pulsed. It can be a current measurement, a potential measurement or a measurement of charge. It can be a steady state measurement, where a current or potential that does not substantially change during the measurement is monitored, or it can be a dynamic measurement, e.g., one in which the rate of current or potential change in a given time period is monitored. When a current is measured it is useful to have a potentiostat in the circuit connecting the implanted sensing electrode and the second electrode, that can be a reference electrode, such as an Ag/AgCl electrode. When a current is measured the reference electrode may serve also as the counter electrode. The counter electrode can also be a separate, third electrode, such as a platinum, carbon, palladium or gold electrode.

A “detection device” is any device or material that allows for the detection of one or more electrical signals internally-generated in the sensor strip. The detection device is generally contacted to a sensor strip at two positions through passive contact of equipotential electrodes. In a specific embodiment, the apparatus includes a housing with a display panel located on the top front or face.

An “electrode” or “lead” is a wire, electrical lead, connection, electrical contact or the like that is attached at one end to a detection unit and contacted at the other end directly or indirectly to a sensor strip. Contact to sensor strip is generally electrically passive in nature and occurs at two positions. One of the electrodes may serve as an electron sink or electrical ground.

“Enzyme biosensors” or “catalytic biosensors” as used herein refer to reaction systems that utilize one or more enzyme types as the macromolecular binding agents and take advantage of the complementary shape of the selected enzyme and the targeted analyte. Enzymes are proteins that perform most of the catalytic work in biological systems and are known for highly specific catalysis. The shape and reactivity of a given enzyme limit its catalytic activity to a very small number of possible substrates. Enzymes are also known for speed, working at rates as high as 10,000 conversions per second per enzyme molecule. Enzyme biosensors rely on the specific chemical changes related to the enzyme/analyte interaction as the means for determining the presence of the targeted analyte. For example, upon interaction with an analyte, an enzyme may generate electrons, a colored chromophore or a change in pH as the result of the relevant catalytic enzymatic reaction. Alternatively, upon interaction with an analyte, an enzyme may cause a change in a fluorescent or chemiluminescent signal that can be recorded by an appropriate detection system.

As used herein, an “epitope” or “antibody recognition sequence” refers to that portion of a polypeptide or chemical compound that is required for binding of a specific antibody. In the present invention, the epitope may be part of the native polypeptide, or may be expressed as a fusion protein that also contains an active redox enzyme.

The “immunoreactivity” of a polypeptide or chemical compound as used herein refers to its ability to generate a response from the immune system or to provoke specific antibody binding.

“Immunosensors” as used herein utilize antibodies as binding agents. Antibodies are protein molecules that bind with specific foreign entities, called antigens, that can be associated with disease states. Antibodies attach to antigens and either remove the antigens from a host and/or trigger an immune response. Antibodies are quite specific in their interactions and, unlike enzymes, they are capable of recognizing and selectively binding to very large bodies such as single cells. Thus, antibody-based biosensors allow for the identification of certain pathogens such as dangerous bacterial strains. As antibodies generally do not perform catalytic reactions, there is a need for special methods to record the moment of interaction between target analyte and recognition agent antibody.

As used herein “polarize” refers to applying a polarized light source to a reaction mixture. Natural sunlight and many sources of artificial light transmit waves whose electric field vectors vibrate in all perpendicular planes with respect to the direction of propagation. When the electric field vectors are restricted to a single plane, then the light is said to be polarized with respect to the direction of propagation. A device used to generate polarized light from unpolarized light is a “polarizer.” A polarizer may be based on one of four physical mechanisms: dichroism, reflection, scattering, and bifringence.

The term “polypeptide” as used herein is used interchangeably with the term “protein” and is defined as a molecule which comprises more than one amino acid subunits. The polypeptide may be an entire protein or it may be a fragment of a protein, such as a peptide or an oligopeptide. The polypeptide may also comprise alterations to the amino acid subunits, such as methylation or acetylation.

A “reaction cell” is a container that comprises components for carrying out the biomarker detection method, and may include a biosensor chip. A reaction cell may comprise a solution of catalase and glucose, a recombinant fusion protein characterized by a redox activity and an immunoreactivity against a capture antibody, and an electrode having immobilized on its surface the capture antibody and a biomolecular peroxide sensor.

A “redox” or “oxidation-reduction” reaction describes any reaction in which electrons are transferred from one molecule to another. The process of oxidation cannot occur without a corresponding reduction reaction. Oxidation must always be “coupled” with reduction, thus the electrons that are “lost” by one substance must always be “gained” by another. Each reaction by itself is called a “half-reaction”. All metal atoms are characterized by their tendency to be oxidized, losing one or more electrons, forming a positively charged ion, called a cation.

The terms “redox-active moiety” or “redox-active species” refers to a compound that can be oxidized and reduced, i.e. which contains one or more chemical functions that accept and transfer electrons.

The term “redox protein” or “redox-active protein” refers to proteins that bind electrons reversibly. The simplest redox proteins, in which no prosthetic group is present, are those that use reversible formation of a disulfide bond between two cysteine residues, as in thioredoxin. Most redox proteins however use prosthetic groups, such as flavins or NAD. Many use the ability of iron or copper ions to exist in two different redox states.

II. Amperometric biosensors

Amperometric enzyme electrodes typically require some form of electrical communication between the electrode and the active site of the redox enzyme that is reduced or oxidized by the substrate. In one type of enzyme electrode, a non-natural redox couple mediates electron transfer from the substrate-reduced enzyme to the electrode. In this scheme, the enzyme is reduced by its natural substrate at a given rate; the reduced enzyme is in turn, rapidly oxidized by a non-natural oxidizing component of a redox couple that diffuses into the enzyme, is reduced, diffuses out and eventually diffuses to an electrode where it is oxidized. Electrons from a substrate-reduced enzyme will be transferred either to the enzyme's natural re-oxidizer or, via the redox-centers of the polymer to the electrode. Only the latter process contributes to the current.

Amperometric detection of redox active molecules in solution is used to detect very small amounts of a substance or chemical in a solution via oxidation or reduction of that chemical, usually at an electrode. This type of analysis is useful in forensic chemistry, clinical chemistry, and many other applications in which a trace amount of material is to be discerned in a solution.

The present invention exploits the use of a redox enzyme that can be immobilized or “wired” onto a screen-printed chip. The substrate chip for the biosensor is a commercially available chip that has horseradish peroxidase incorporated into the carbon dye of the working electrode (produced by Cambridge Life Sciences, Cambridge, UK). These chips, in their unmodified form, act as peroxide sensors, and the incorporation of the horseradish peroxidase in combination with the electrode comprises a “biomolecular peroxide sensor”. Any number of available amperometric peroxide sensors would be valid starting substrates for the design. In the present biosensor design, several layers of molecular components are incorporated onto these peroxide sensors, converting them into sensors that selectively measure the concentration of a single protein of interest.

The chip of the present invention represents a substantial improvement over other amperometric immunosensors. Analyte binding to an immobilized antibody at the working electrode surface is detected by current flow to an immobilized redox enzyme (hydrogen peroxidase) at low voltage. Analyte concentration is inversely related to current flow in this model. This is a competitive assay mechanism that does not require stirring or wash steps that can complicate a handheld device.

The biosensor design depends critically on a custom-designed signal transduction molecule. The present invention utilizes a gene encoding glucose oxidase from Penicillium amagasakiense. This molecule is a recombinant fusion protein constructed in vitro, expressed in yeast and purified for application to the chip. Glucose oxidase (Gox) is an enzyme that generates hydrogen peroxide from glucose and provides the enzymatic core for the novel fusion protein. However, any functional glucose oxidase which may be used in conjunction with the biomolecular peroxide sensor is contemplated in the present invention. Thus, the epitopes of the present invention may be fused to any of a number of contemplated glucose oxidase polypeptides, such as SEQ ID NO:2 or SEQ ID NO:3.

To adapt the biosensor of the present invention to detect a protein, a monoclonal antibody is identified that recognizes this protein of interest (the capture antibody)and is biotinylated. The peptide epitope recognized by this antibody is then mapped. One with skill in the art recognizes that this can be accomplished using a variety of standard techniques. Once the epitope is known, an analyte-Gox fusion protein is engineered by inserting DNA encoding this epitope peptide into the coding sequence of Gox. This construct is transfected into yeast, which produce the fusion protein and secrete it into the culture media from which the fusion protein is purified. This fusion protein shares two important characteristics: (a) enzymatic activity derived from Gox and (b) immunoreactivity with the capture antibody. In essence, this fusion protein provides the signal transduction machinery to convert binding of the protein of interest into hydrogen peroxide, which can be measured electrochemically by the chip.

The fusion protein is integrated into the biosensor by modifying the chip substrate in two ways: (1) by directly immobilizing the capture antibody at the electrode surface through an avidin-biotin interaction, and (2) by incorporating a catalase scavenger system in bulk solution. These modifications divide the chip into two distinct microenvironments. Only fusion protein localized to the microenvironment at the working electrode surface should generate an electrical signal. Any hydrogen peroxide generated from unbound fusion protein is consumed by the catalase in bulk solution and does not generate a signal.

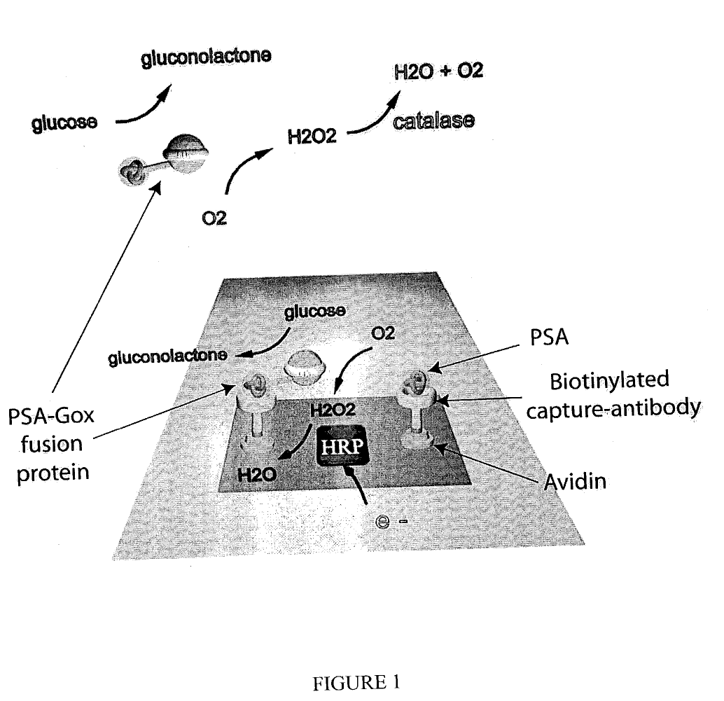

The reaction mechanism, as demonstrated in FIG. 1, can be described as follows. The protein analyte (X) competes with the fusion protein (X-Gox) for binding to the capture antibody at the electrode surface. After binding for five minutes, the chip is polarized to +50 mV and glucose is added. If no protein X is present, all the capture antibody sites are occupied by fusion protein, which generates hydrogen peroxide upon addition of glucose. This hydrogen peroxide is broken down by the immobilized peroxidase in the working electrode. In order to regenerate this redox enzyme, a current flows which can be measured by a simple potentiostat. Current magnitude in this system is in the several hundred-nanoampere range. Again, peroxide produced by unbound fusion protein in bulk solution is hydrolyzed by catalase and cannot reach the working electrode to generate a signal. If protein X exists in the test solution (for example, a drop of whole blood), it competes for binding to the lawn of capture antibody with the fusion protein. Fusion protein displaced by the binding of protein X can no longer generate a current signal. Therefore, the global current flowing to the chip decreases in a fashion directly related to the concentration of protein X in the test solution.

An embodiment of the present invention is an inexpensive handheld device based on this type of biosensor chip. This device would serve in a similar fashion to currently available portable glucometers used by diabetics to follow their blood sugar. As demonstrated by a variety of commercial glucometers, simple potentiostats can be manufactured inexpensively in a very small size. This device would accept disposable protein biosensor chips of the present invention. A drop of a body fluid would be applied to the biosensor and analyte concentration, based on the generated current, would be available in less than 15 minutes.

Several characteristics of this type of system are advantageous for a point-of-service PSA sensor. (1) The time to generate test results is short (10-15 minutes); (2) The reaction mechanism requires no stirring or washing steps which would significantly complicate a handheld device; (3) The hardware required for assay is, therefore, only a simple potentiostat capable of measuring current in the 50 to 500 nanoampere range (this magnitude of current does not require a Faraday cage or other sophisticated shielding equipment which would invalidate this assay's use in an inexpensive handheld device); (4) The low voltage used for the assay (+50 mV) is advantageous since most proteins in body fluids will not be electrochemically active at this voltage; (5) Through the use of a recombinant approach and simple purification methodology, production of large quantities of the fusion protein can be performed simply and economically; (6) The fusion protein is quite stable and resistant to pH changes, allowing for good storage characteristics; (7) The chip substrates, themselves, are stable for 18 months at 4° C., protected from light; (8) The molecular biology used to produce this fusion protein is designed to facilitate ease in swapping of both the capture antibody and epitope domain, allowing the sensor to easily be adapted to detect other proteins.

III. Immunological Reagents

In certain aspects of the invention, one or more antibodies may be produced to the desired epitope. The epitope may comprise a biomarker or tumor marker These antibodies may be used in various diagnostic or therapeutic applications, described herein below.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The methods for generating monoclonal antibodies (MAbs) generally begin along the same lines as those for preparing polyclonal antibodies. Briefly, a polyclonal antibody is prepared by immunizing an animal with a LEE or CEE composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific stimulators of the immune response, known as adjuvants. Suitable adjuvants include all acceptable immunostimulatory compounds, such as cytokines, chemokines, cofactors, toxins, plasmodia, synthetic compositions or LEEs or CEEs encoding such adjuvants.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages, but mice are preferred, with the BALB/c mouse being most preferred as this is most routinely used and generally gives a higher percentage of stable fusions.

The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bu1; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. The use of electrically induced fusion methods is also appropriate.

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. In another example, LEEs or CEEs can be used to produce antigens in vitro with a cell free system. These can be used as targets for scanning single chain antibody libraries. This would enable many different antibodies to be identified very quickly without the use of animals.

Alternatively, monoclonal antibody fragments encompassed by the present invention can be synthesized using an automated peptide synthesizer, or by expression of full-length gene or of gene fragments in E. coli.

IV. Antibody Conjugates

The present invention further provides antibodies that are conjugated. The antibodies are generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art.

A type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Molecules containing azido groups may also be used to form covalent bonds to proteins through reactive nitrene intermediates that are generated by low intensity ultraviolet light. In particular, 2- and 8-azido analogues of purine nucleotides have been used as site-directed photoprobes to identify nucleotide binding proteins in crude cell extracts. The 2-and 8-azido nucleotides have also been used to map nucleotide binding domains of purified proteins and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulthydryl groups in the Fe region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fe region have also been disclosed in the literature. This approach has been reported to produce diagnostically and therapeutically promising antibodies which are currently in clinical evaluation.

V. Immunodetection Methods

In still further embodiments, the present invention concerns immunodetection methods for binding, purifying, removing, quantifying and/or otherwise generally detecting biological components such as biomarker epitopes or fusion proteins containing biomarker epitopes, as described by the present invention.

In general, the immunobinding methods include obtaining a sample suspected of containing the epitope of interest in an expressed message and/or protein, polypeptide and/or peptide, and contacting the sample with a first anti-epitope message and/or anti-epitope translated product antibody in accordance with the present invention, as the case may be, under conditions effective to allow the formation of immunocomplexes.

The immunobinding methods also include methods for detecting and quantifying the amount of an antigen component in a sample and the detection and quantification of any immune complexes formed during the binding process. Here, one would obtain a sample suspected of containing an antigen, and contact the sample with an antibody against the epitope-containing antigen, and then detect and quantify the amount of immune complexes formed under the specific conditions.

In terms of antigen detection, the biological sample analyzed may be any sample that is suspected of containing an antigen, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum, although tissue samples or extracts are preferred.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any epitope-containing antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

The immunodetection methods of the present invention have evident utility in the diagnosis and prognosis of conditions such as various diseases wherein a specific biomarker is expressed. Here, a biological and/or clinical sample suspected of containing a specific disease associated biomarker is used.

In the clinical diagnosis and/or monitoring of patients with various forms a disease, such as, for example, cancer, the detection of a cancer specific biomarker, and/or an alteration in the levels of a cancer specific gene product, in comparison to the levels in a corresponding biological sample from a normal subject is indicative of a patient with cancer. However, as is known to those of skill in the art, such a clinical diagnosis would not necessarily be made on the basis of this method in isolation. Those of skill in the art are very familiar with differentiating between significant differences in types and/or amounts of such biomarkers, which represent a positive identification, and/or low level and/or background changes of the biomarkers. Indeed, background expression levels are often used to form a “cut-off” above which increased detection will be scored as significant and/or positive. Of course, the antibodies of the present invention in any immunodetection or therapy known to one of ordinary skill in the art.

VI. Epitopic Core Sequences

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes.

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective. For instance, 18 of 20 peptides designed according to these guidelines, containing 8-39 residues covering 75% of the sequence of the influenza virus hemagglutinin HAI polypeptide chain, induced antibodies that reacted with the HAI protein or intact virus; and 12/12 peptides from the MuLV polymerase and 18/18 from the rabies glycoprotein induced antibodies that precipitated the respective proteins.

U.S. Pat. No. 4,554,101, incorporated herein by reference, teaches the identification and/or preparation of epitopes from primary amino acid sequences on the basis of hydrophilicity. Through the methods disclosed in Hopp, one of skill in the art would be able to identify epitopes from within an amino acid sequence.

Numerous scientific publications have also been devoted to the prediction of secondary structure, and/or to the identification of epitopes, from analyses of amino acid sequences. Any of these may be used, if desired, to supplement the teachings of Hopp in U.S. Pat. No. 4,554,101.

Moreover, computer programs are currently available to assist with predicting antigenic portions and/or epitopic core regions of proteins. Examples include those programs based upon the Jameson-Wolf analysis, the program PepPlot®, and/or other new programs for protein tertiary structure prediction. Another commercially available software program capable of carrying out such analyses is MacVector (IBI, New Haven, Conn.).

Antigenic epitope-bearing peptides and polypeptides of the invention are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody reactive with the native protein.

Antigenic epitope-bearing peptides and polypeptides of the invention designed according to the above guidelines preferably contain a sequence of at least seven, more preferably at least nine and most preferably between about 15 to about 30 amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

The epitope-bearing peptides and polypeptides of the invention may be produced by any conventional means for making peptides or polypeptides including recombinant means using nucleic acid molecules of the invention. For instance, a short epitope-bearing amino acid sequence is fused to a larger polypeptide which acts as a carrier during recombinant production and purification, as well as during immunization to produce anti-peptide antibodies.

Immunogenic epitope-bearing peptides of the invention, i.e., those parts of a protein that elicit an antibody response when the whole protein is the immunogen, are identified according to methods known in the art.

In further embodiments, major antigenic determinants of a polypeptide may be identified by an empirical approach in which portions of the gene encoding the polypeptide are expressed in a recombinant host, and/or the resulting proteins tested for their ability to elicit an immune response. For example, PCR™ can be used to prepare a range of peptides lacking successively longer fragments of the C-terminus of the protein. The immunoactivity of each of these peptides is determined to identify those fragments and/or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTs™ system (Genosys Biotechnologies, Inc., The Woodlands, Tex.). In this method, overlapping peptides are synthesized on a cellulose membrane, which following synthesis and/or deprotection, is screened using a polyclonal and/or monoclonal antibody. The antigenic determinants of the peptides which are initially identified can be further localized by performing subsequent syntheses of smaller peptides with larger overlaps, and/or by eventually replacing individual amino acids at each position along the immunoreactive peptide.

Once one and/or more such analyses are completed, polypeptides are prepared that remove and/or add at least the essential features of one and/or more antigenic determinants. The peptides are then employed in the methods of the invention to reduce and/or enhance the production of antibodies when isolated protein and/or gene constructs made by the methods of the present invention is administered to a mammal, preferrably a human. Minigenes and/or gene fusions encoding these determinants can also be constructed and/or inserted into expression vectors by standard methods, for example, using PCR™ cloning methodology.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

VII. Fusion Proteins

A specialized kind of insertional variant is the fusion protein. This molecule generally has all or a substantial portion of the native molecule, linked at the N- or C-terminus, to all or a portion of a second polypeptide. In the present invention, a fusion may comprise a biomarker epitope sequence and a Gox sequence. Inclusion of a cleavage site at or near the fusion junction will facilitate removal of an extraneous polypeptide that is used as a tag to facilitate purification. Other useful fusions include linking of functional domains, such as active sites from enzymes such as a hydrolase, glycosylation domains, cellular targeting signals or transmembrane regions.

Following transduction with an expression construct or vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference.

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses cell lines, which are transfected with an expression construct or vector that expresses a therapeutic protein such as a tumor suppressor. Examples of mammalian host cell lines include Vero and HeLa cells, other B- and T-cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thyindine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large-scale production of cells and cell products. However, suspension cultured cells have limitations, such as tumorigenic potential and lower protein production than adherent cells.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

Selection of an anti-PSA monoclonal antibody and characterization of the recognition peptide

To adapt the biosensor design to the detection of PSA, a monoclonal anti-PSA capture antibody was selected. This antibody was selected for several characteristics: (a) it was available in purified form without bovine serum albumin and, hence, could be biotinylated without difficulty; (b) it was capable of recognizing PSA after reduction and denaturation, indicating that it recognizes a linear epitope within the primary structure of PSA.

Selection of an antibody for detection of PSA on the biosensor chip is a critical step in the production process. Monoclonal antibodies are contemplated. This antibody should bind to total PSA and should not cross react with human glandular kallikrein 2 (HK2), which shares 80% sequence homology to PSA. Also note that the final biosensor design depends on production of a fusion protein that shares glucose oxidase enzymatic activity and PSA immunoreactivity.

Because the recognition peptide for the antibody had not previously been described, it was necessary to map this domain. A library of overlapping peptides was synthesized corresponding to the entire sequence of human PSA (produced by Mimotopes, San Diego, Calif.). The capture antibody was reacted with this library and the pattern of reacting peptides was used to define a seven amino-acid recognition epitope. A synthetic peptide was then prepared corresponding to this epitope. Experiments using dot blots confirmed that the capture antibody bound to this specific peptide with high affinity.

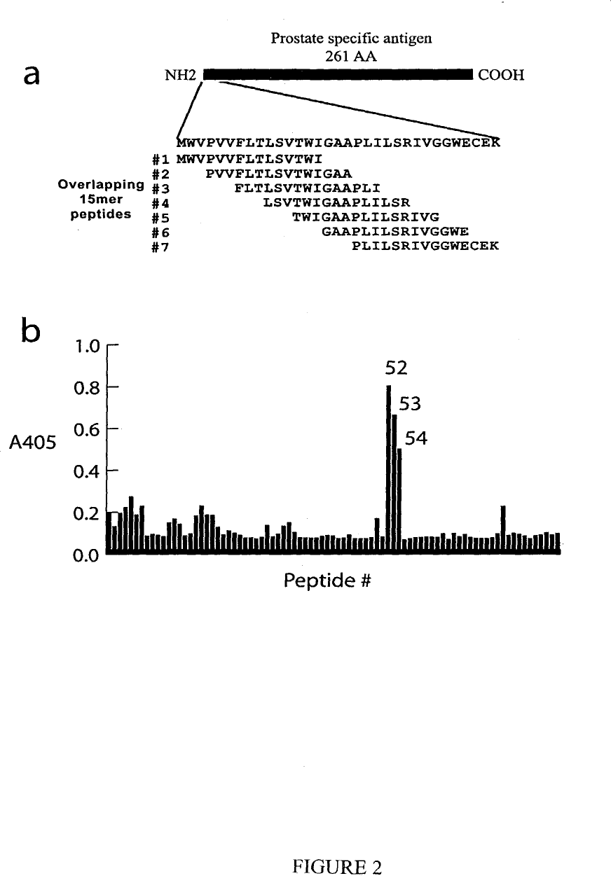

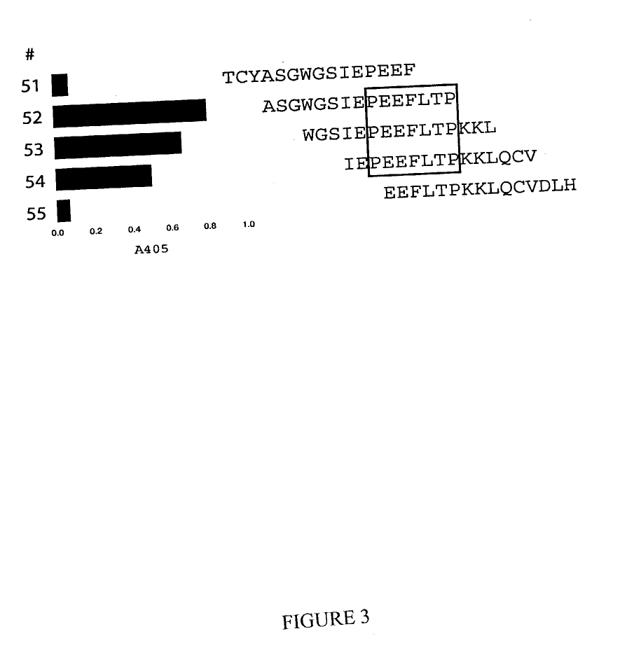

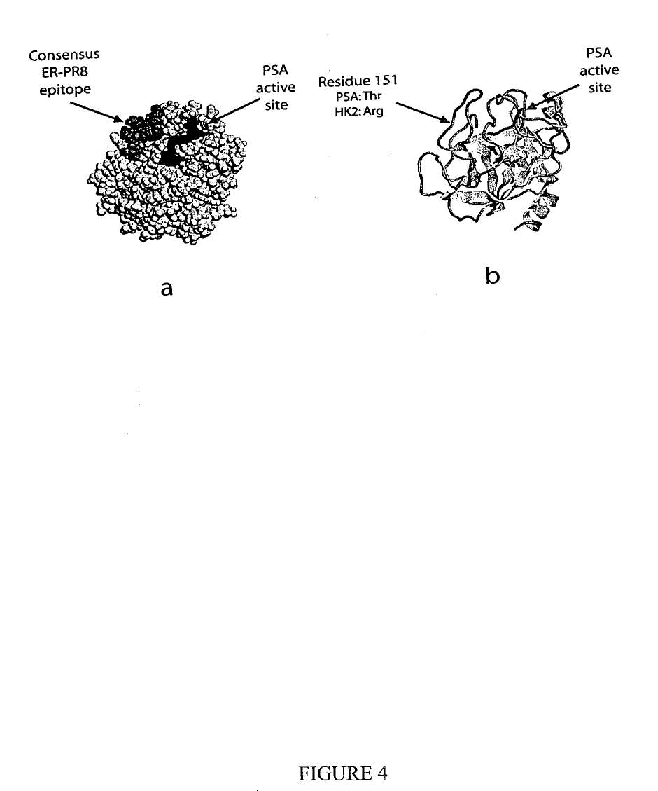

Both the peptide and recombinant approaches depend upon knowledge of the peptide sequence bound by the ER-PR8 antibody. Previous attempts to map the epitope of this antibody through competition with other antibodies for known binding sites on PSA have been unsuccessful. As an alternative strategy, a library of overlapping fifteen amino-acid peptides spanning the entire sequence of PSA was prepared (FIG. 2). Screening of these peptides using an ELISA-based assay demonstrated three overlapping peptides with significant binding to the antibody (peptides 52-54; FIG. 2). Closer examination of the overlaps identified a consensus 7 amino-acid peptide epitope (NH2-PEEFLTP-COOH; FIG. 3). Mapping these residues onto the predicted three-dimensional structure of PSA demonstrated three important characteristics (FIG. 4). First, the critical amino acids in the predicted epitope are located at the protein surface, where they form a prominent ridge. Second, this peptide is conserved in HK2 except for a single nonconservative (Thr to Arg) substitution at position 151. Finally, the peptide is located close to the active site of the enzyme. These characteristics strongly support that this peptide represents the epitope for ER-PR8 since this antibody binds to native PSA, does not bind to HK2 and inhibits PSA enzymatic activity by 46%.

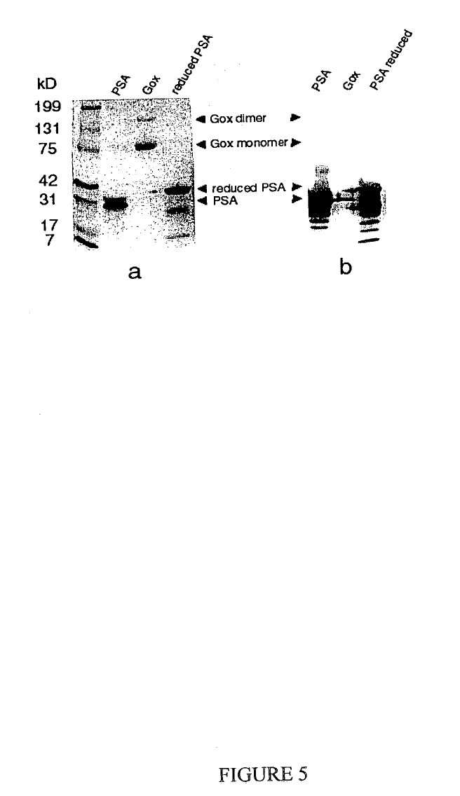

Several different anti-PSA monoclonal antibodies were tested on dot blots with native, denatured and reduced PSA protein. The antibody selected was clone ER-PR8. The purified and biotinylated antibody is available from several commercial sources. It binds to total PSA and does not recognize HK2. Moreover, ER-PR8 recognizes denatured and reduced full length PSA as well as some clipped PSA fragments on western blots analysis (FIG. 5). These results are consistent with published observations, confirm that ER-PR8 binding is not dependent on the tertiary structure of PSA and suggest that its recognition epitope is continuous.

### Example 2

Cloning, mutagenesis and prokaryotic expression of recombinant glucose oxidase from Penicillium amagasakiense and preparation of a PSA epitope-containing fusion protein

Highly homologous glucose oxidase genes have been cloned from Penicillium amagasakiense and Aspergillus niger. Moreover, the wild-type Penicillium gene has been successfully expressed in Escherichia coli at high levels. Although this protein accumulates in insoluble inclusion bodies, Kaliz et al. were successful in regenerating functionally active enzyme using an in vitro system for refolding the protein. Other notable advantages of the Penicillium protein over its Aspergillus homolog are its higher turnover rate and better affinity for Beta-D-glucose, which translates into greater sensitivity for a biosensor based on this enzyme. For these reasons, Penicillium protein was chosen as the basis for the recombinant strategy to produce rPSA-Gox.

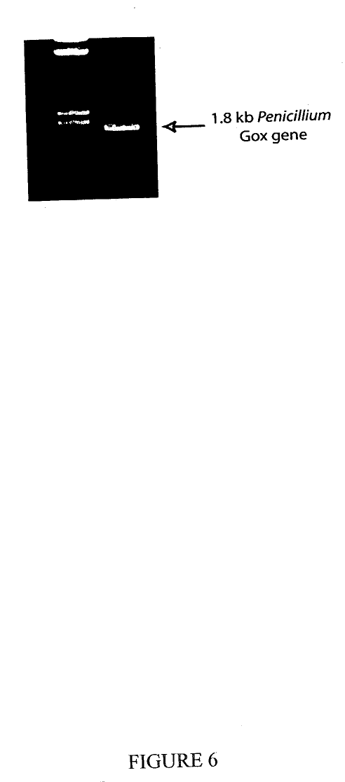

To isolate the gene encoding Penicillium Gox, PCR primers were designed based on the published Gox sequence to allow amplification from genomic DNA. These primers also mutated the wild-type gene to add an amino-terminal HAT tag. The HAT tag, a 19-amino acid sequence derived from the chicken lactate dehydrogenase protein, contains multiple histidines which should allow purification of the recombinant protein in a single step using immobilized metal ion affinity chromatography (IMAC). The tag was placed at the N-terminus because (1) this region is the most divergent between Penicillium and Aspergillus Gox; and (2) the amino-terminus does not encroach on the active site or the homodimer contact points visible in the three-dimensional crystal structure of Gox. The second mutation introduced into the Gox gene was the placement of unique restriction sites downstream from the HAT tag to allow cassettes encoding custom epitopes (such as PSA) to be easily swapped into the recombinant Gox protein. After preparing genomic DNA from Penicillium amagasakiense (ATCC strain 28686), these primers were used to amplify the full length 1800 bp PCR fragment encoding the mutated Gox gene (FIG. 6), which encodes SEQ ID NO:2, GenBank Accession No. P81156.

Enzymatically active Aspergillus glucose oxidase (SEQ ID NO:3 GenBank Accession No. CAC12802) has previously been expressed at high levels (>300 ug/mL) in Saccharomyces cerevisiae. Pichia pastoris EasySelect System (Invitrogen Corp., Carlsbad, Calif.) was used for these experiments since Pichia has been shown to express a variety of recombinant proteins at significantly higher levels than Saccharomyces. The Penicillium Gox gene was inserted into a Pichia expression vector under the control of the alcohol oxidase promoter and the previously defined consensus peptide epitope recognized by the biosensor capture antibody (ER-PR8) was inserted at the carboxyl terminus. In addition, an extension of six sequential histidine residues was added downstream of this epitope to facilitate purification using immobilized metal affinity chromatography (IMAC). Note that this configuration differs significantly from the prokaryotic expression vector described above, in which the epitope tag was placed at the N-terminus. The decision to place the tags in the carboxyl terminus in this new mutant was based on protein folding considerations and on a careful examination of the three-dimensional crystal structure of glucose oxidase. Within this structure, the carboxyl terminus is well away from the active site and monomer-monomer contact points, suggesting that it would be a relatively innocuous location for mutagenesis. The completed vector (pPicZ-GPM6) was designed to drive secretion of the recombinant PSA-Gox fusion protein (GPM6) into the media under the control of the native Gox signal sequence.

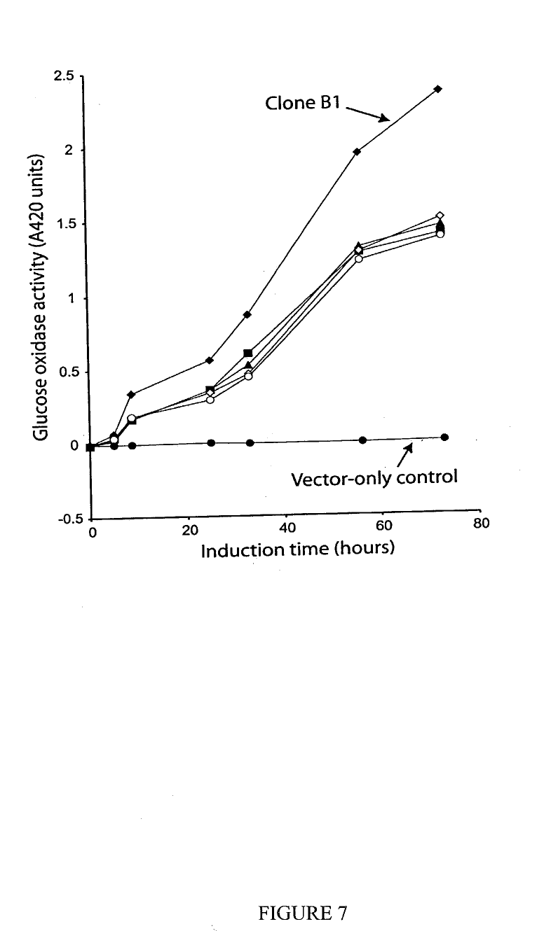

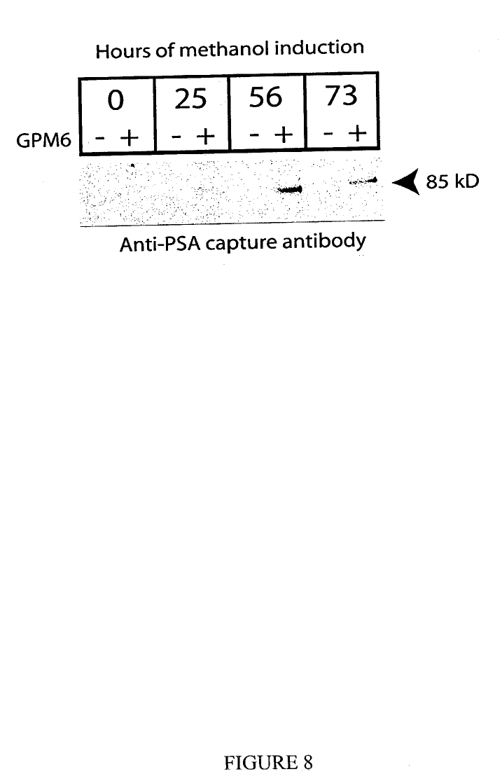

pPicZ-GPM6 was transfected into Pichia pastoris strain X-33 and Mut⁺clones were isolated. These clones were screened for the presence of the Gox sequence by PCR and positive clones were expanded for small-scale expression studies. Clones were induced with methanol and conditioned media was collected at varying time points and assayed for Gox activity by spectrophotometric assay. As shown in FIG. 7, conditioned media from several clones contained functional Gox activity. A single clone (B1) showed ˜2-fold activity as compared to the other clones and was selected for further larger-scale expression studies. Western blot analysis of B1-GPM6 culture supernatants clearly shows the accumulation of a recombinant protein recognized by the ER-PR8 anti-PSA capture antibody (FIG. 8). These results confirm several points critical to the success of the project: (1) Pichia are capable of expressing, properly processing and secreting Penicillium Gox into the culture media; (2) The mutations introduced into the recombinant PSA-Gox fusion protein (GPM6) do not compromise protein folding or eliminate enzymatic activity; and (3) The position of the buried PSA epitope within the primary sequence of GPM6 does not block binding of the capture antibody to denatured fusion protein.

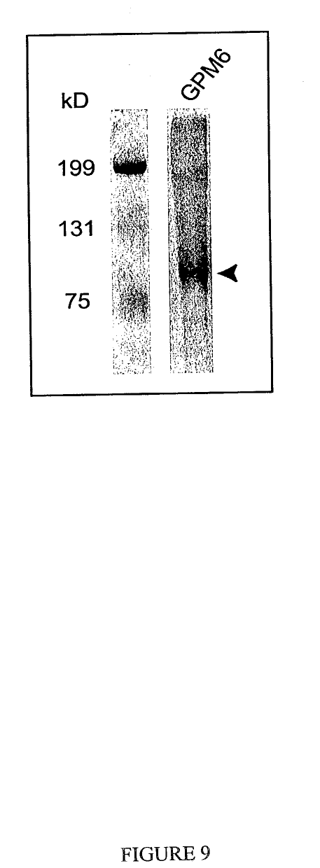

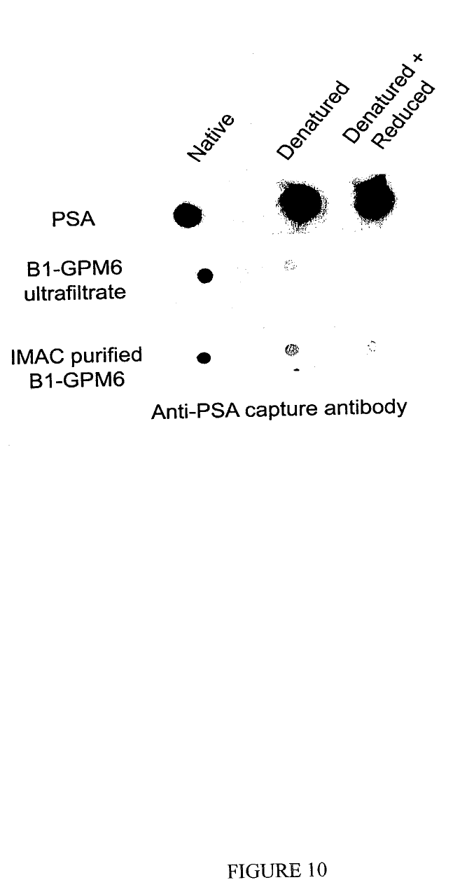

B1-GPM6 production was then increased to 1 liter scale and a small test aliquot was subjected to the following test purification protocol. The material was initially ultrafiltrated using a tangential flow system with 10 kD cutoff membranes and then diluted 10-fold in neutral high salt binding buffer. After binding to a Nickel column and extensive washing, enzymatically active GPM6 was eluted using a pH step gradient. Fractions containing Gox activity were pooled, re-concentrated by ultrafiltration and gently alkalinized to pH 6.0. SDS-PAGE analysis identified a single diffuse band at ˜85-90 kD, likely representing GPM6 monomers with heterogeneous glycosylation (FIG. 9). To confirm that the PSA epitope was not masked by the three-dimensional conformation of the folded fusion protein, qualitative dot blot analysis was performed on the purified material. These dot blots demonstrated that the capture antibody bound well to natively folded GMP6 as well as to denatured and reduced protein as described previously (FIG. 10).

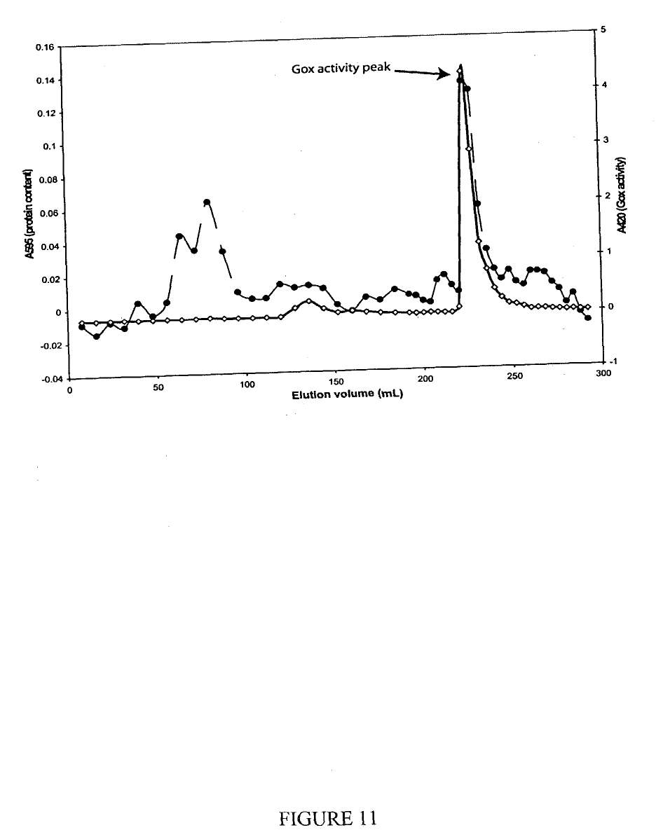

A series of experiments demonstrated that functional recombinant Gox can be purified with a single inexpensive anion-exchange chromatography step (FIG. 11), a single liter of Pichia culture supernatant contains enough active fusion protein for 600 to 800 biosensor chip assays.

A synthetic DNA adapter was designed that encoded this peptide and introduced it into the carboxyl terminus of the Gox coding sequence from Penicillium amagasakiense. The mutant coding sequence of the fusion protein (called GPM6) was introduced into a vector designed for expression in the methyltrophic yeast, Pichia pastoris. The Pichia EasySelect expression system (Invitrogen Corp., Carlsbad, Calif.) was used for these studies, although as variety of yeast-based expression systems would be applicable. Finally, the construct was designed such that the novel fusion protein should be secreted into the culture media under the control of the native signal sequence present in the Gox coding sequence. GPM6 was transfected into the Pichia and transformants were isolated. Numerous clones were identified that secreted GPM6 into the media which had high levels of Gox activity. In addition, western analysis of the culture media demonstrated that the anti-PSA capture antibody recognized GPM6. This result confirmed that burying the epitope within the coding sequence of Gox did not block recognition by the capture antibody. The fusion protein was then purified in an active form in two steps: (a) ion-exchange chromatography using a DEAE-Sepharose fast flow column, followed by (b) desalting and alkalinization using size-exclusion chromatography. A single liter of Pichia culture produces enough fusion protein for >600 biosensor assays.

### Example 3

### Preparation of protein biosensor chips

Unmodified peroxide sensor chips (Cambridge Life Sciences, Cambridge, UK) were incubated with deglycosylated avidin overnight at 4° C. to adsorb the avidin to the working electrode surface. After blocking with 3% caseine, the chips were washed with phosphate buffered saline (PBS) and then incubated with the biotinylated capture antibody overnight at 4° C. After a second wash with PBS, the chips were either dried for storage or stored under PBS until used. To perform the biosensor assay, chips were placed into a custom electrochemical cell attached to a potentiostat. The sample to be tested is mixed with GPM6 fusion protein and allowed to incubate with the chips for five minutes. PBS containing catalase and 1% glucose is then added to the chips, which are polarized to +50 mV, and the current is measured against time. The concentration of PSA in the test solution is calculated from this current response.

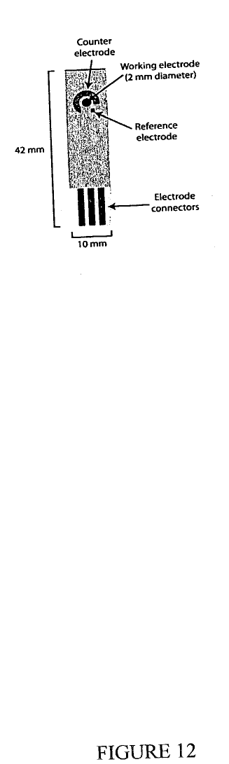

The starting chip for these experiments is screen-printed by Cambridge Life Sciences, UK and has a three-electrode configuration (FIG. 12) with a working electrode of 2 mm diameter. The working electrode is made of carbon dye into which horseradish peroxidase (HRP) has been added to “wire” the enzyme directly into the electrode. When the working electrode is held at a potential of +50 mV Vs. Ag/AgCl, this unmodified chip can detect hydrogen peroxide in solution. After HRP catalyzes the breakdown of hydrogen peroxide to water and oxygen, the redox enzyme regenerates by accepting an electron from the working electrode. This results in a measurable current flow that can be detected with a simple potentiostat.

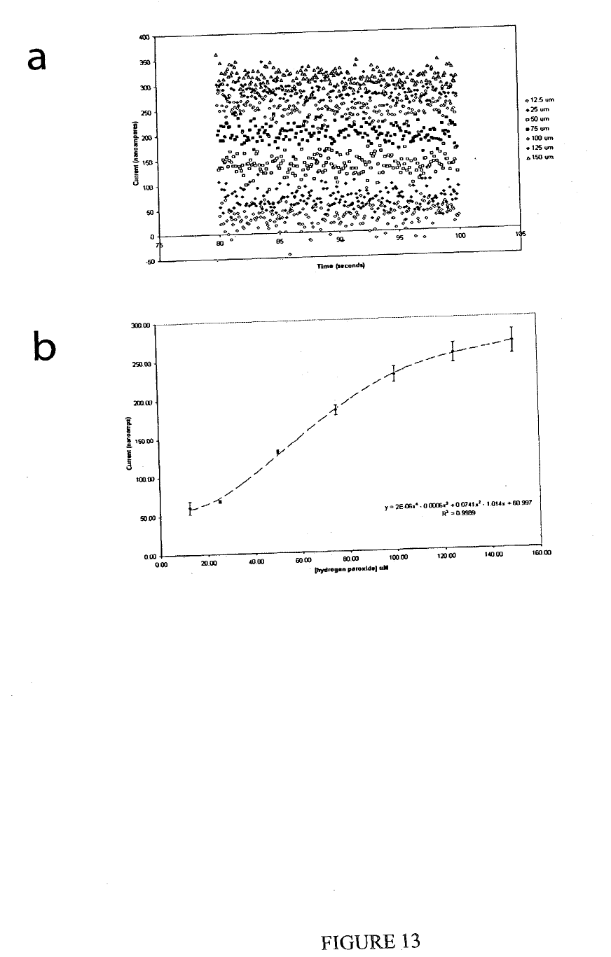

The current response of the chip under these conditions increases with rising concentration of substrate (hydrogen peroxide) present in solution (see FIG. 13). FIG. 13 demonstrates that this response is linear over a concentration range of 25 to 100 micromolar (uM); above which the current response starts to plateau. The current response for a single chip exposed to either 50 or 100 μM H₂O₂ was 132.7+/−0.6 and 242.2+/−2.3 nanoamperes (nA; n=6 measurements per chip), respectively. As expected, interchip variability in absolute current response was slightly greater (50 uM: 130.6+/−2.4 nA; 100 uM: 228.1+/−9.9 nA; n=5).

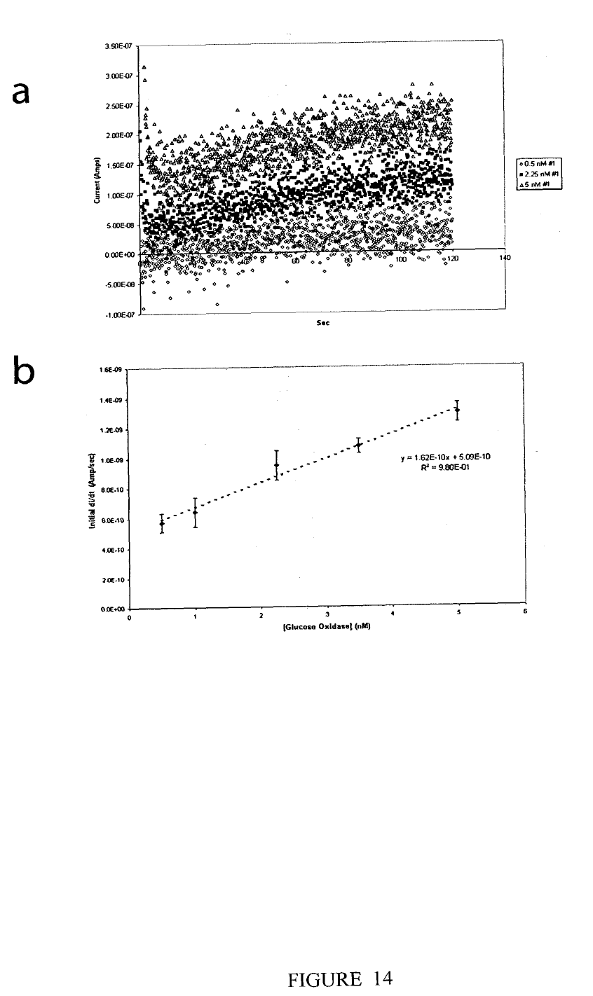

Increasing concentrations of purified glucose oxidase to the biosensor chip were added in the presence of glucose. FIG. 14 shows the current response to glucose oxidase and demonstrates a rising current over time consistent with the activity of the enzyme. The slope of the i-t curve (di/dt) was calculated over a time interval of 15 to 60 seconds following addition of glucose oxidase. During this time interval, the rise in current was linear since substrate concentration was not limiting. FIG. 14 confirms that the di/dt₁₅₋₆₀ has the expected linear relationship with enzyme concentration. Therefore, di/dt₁₅₋₆₀ can be used to measure active glucose oxidase in a solution of unknown concentration.

### Example 4

The combination of adsorbed surface avidin and a catalase scavenging system results in two distinct chip microenvironments (surface vs. bulk solution)

The PSA biosensor chip design depends on the existence of two distinct microenvironments (the working electrode surface and bulk solution). These microenvironments are set up artificially by immobilizing the capture antibody at the electrode surface with an avidin-biotin interaction and by including excess catalase in solution to scavenge peroxide produced by glucose oxidase outside the surface microenvironment. Wright et al. originally used catalase for this purpose in a biosensor that detected biotin. Subsequently, a similar system was used to detect the herbicide atrazine. The chip design for the atrazine system differs from that of the proposed PSA biosensor, since antibodies were not directly attached to the working electrode.

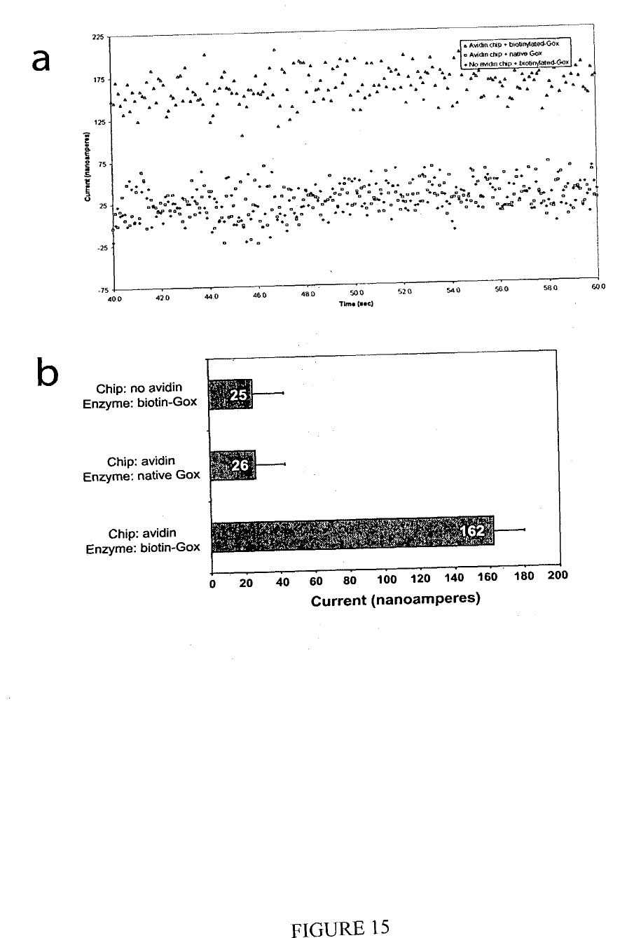

The validity of this system was tested with the following experiment. Deglycosylated avidin was adsorbed to the surface of the biosensor chip and assays were performed in PBS containing 1% glucose and 0.5 mg/mL catalase. Addition of 16 picomoles (pmols) of biotinylated-glucose oxidase resulted in a current response of 162 nA. Native glucose oxidase, which lacks the biotin tag and should not be preferentially localized at the surface , microenviromnent, produced only a background current of 25 nA. Consistent with this result, only background current was observed when biotinylated-Gox was forced to remain in bulk solution on chips lacking adsorbed avidin. A representative experiment of this type is shown in FIG. 15. These results support the fact that two distinct microenvironments exist on the biosensor chip and that only glucose oxidase concentrated at the surface will generate a significant current response.

### References

All patents and publications mentioned in the specification are indicative of the level of those skilled in the art to which the invention pertains. All patents and publications are herein incorporated by reference to the same extent as if each individual publication was specifically and individually indicated to be incorporated by reference.

U.S. patents
5,593,852
5,698,089
5,567,301
5,478,766
4,789,804
5,485,277
5,492,840
5,268,305
5,200,051
5,212,050
4,196,265
4,938,948
5,196,066
4,554,101
5,846,744
5,094,951
5,266,688
6,214,205
6,197,534
6,171,238
6,121,009
6,110,696
6,100,045
6,212,417
5,972,199
4,683,293
4,808,537
4,812,405
4,818,700
4,837,148
4,855,231
4,857,467
4,879,231
4,882,279
4,885,242
4,895,800
4,929,555
5,002,876
5,004,688
5,032,516
5,122,465
5,135,868
5,166,329
3,817,837
3,850,752
3,939,350
3,996,345
4,277,437
4,275,149
4,366,241
4,472,509
4,938,948

Non-patent literature

Becker C, Wigheden I, Lilja H: Characterization of epitope structure for 53 monoclonal antibodies against prostate-specific antigen. Tumour Biol 20:13, 1999

Clark, L. & Lyons, C. Electrode systems for continuous monitoring in cardiovascular surgery. Ann N Y Acad Sci 102, 29-45 (1962).

Coombs G S, Bergstrom R C, Pellequer J L, et al: Substrate specificity of prostate-specific antigen (PSA). Chem Biol 5:475, 1998

Fowler, J. E., Jr., Bigler, S. A., Bowman, G. & Kilambi, N. K. Race and cause specific survival with prostate cancer: influence of clinical stage, Gleason score, age and treatment. J Urol 163, 137-42 (2000).

Frederick K R, Tung J, Emerick R S, et al: Glucose oxidase from Aspergillus niger. Cloning, gene sequence, secretion from Saccharomyces cerevisiae and kinetic analysis of a yeast- derived enzyme [published erratum appears in J Biol Chem 1990 Jul. 5;265(19):11405]. J Biol Chem 265:3793, 1990

Gallee M P, Visser-de Jong E, van der Korput J A, et al: Variation of prostate-specific antigen expression in different tumour growth patterns present in prostatectomy specimens. Urol Res 18:181, 1990

Ghindilis, A. L., Atanasov, P., Wilkins, M. & Wilkins, E. Immunosensors: electrochemical sensing and other engineering approaches. Biosens Bioelectron 13, 113-31 (1998).

Gizeli, E. & Lowe, C. R. Immunosensors. Curr Opin Biotechnol 7, 66-71 (1996).

Jaremko, J. & Rorstad, O. Advances toward the implantable artificial pancreas for treatment of diabetes. Diabetes Care 21, 444-50 (1998).

Keay, R. W. & McNeil, C. J. Separation-free electrochemical immunosensor for rapid determination of atrazine. Biosens Bioelectron 13, 963-70 (1998).

Koch, S., Wolf, H., Danapel, C. & Feller, K. A. Optical flow-cell multichannel immunosensor for the detection of biological warfare agents. Biosens Bioelectron 14, 779-84 (2000).

Lowe, C. R. Chemoselective biosensors. Curr Opin Chem Biol 3, 106-11 (1999)

Morgan, C. L., Newman, D. J. & Price, C. P. Immunosensors: technology and opportunities in laboratory medicine. Clin Chem 42, 193-209 (1996).

Nilsson O, Andersson I, Peter A, et al: Characterization of antibodies to prostate-specific antigen. Tumour Biol 20:43, 1999

Stenman U H, Paus E, Allard W J, et al: Summary report of the TD-3 workshop: characterization of 83 antibodies against prostate-specific antigen. Tumour Biol 20:1, 1999

Villoutreix B O, Getzoff E D, Griffin J H: A structural model for the prostate disease marker, human prostate-specific antigen. Protein Sci 3:2033, 1994

Wang T J, Linton H J, Rittenhouse H G, et al: Western blotting analysis of antibodies to prostate-specific antigen: cross-reactivity with human kallikrein-2. Tumour Biol 20:75, 1999

Wang T J, Linton H J, Sokoloff R L, et al: Western blotting analysis of antibodies to prostate-specific antigen: specificities for prostate-specific antigen and prostate-specific antigen fragments. Tumour Biol 20:79, 1999

Witt S, Singh M, Kalisz H M: Structural and kinetic properties of nonglycosylated recombinant Penicillium amagasakiense glucose oxidase expressed in Escherichia coli. Appl Environ Microbiol 64:1405, 1998

Witt S, Wohlfahrt G, Schomburg D, et al: Conserved arginine-516 of Penicillium amagasakiense glucose oxidase is essential for the efficient binding of beta-D-glucose. Biochem J 347:553, 2000

Wohlfahrt G, Witt S, Hendle J, et al: 1.8 and 1.9 A resolution structures of the Penicillium amagasakiense and Aspergillus niger glucose oxidases as a basis for modelling substrate complexes. Acta Crystallogr D Biol Crystallogr 55:969, 1999

Wright J D, Rawson K M, Ho W O, et al: Specific binding assay for biotin based on enzyme channelling with direct electron transfer electrochemical detection using horseradish peroxidase. Biosens Bioelectron 10:495, 1995

Yoshitake S, Yamada Y, Ishikawa E, et al: Conjugation of glucose oxidase from Aspergillus niger and rabbit antibodies using N-hydroxysuccinimide ester of N-(4-carboxycyclohexylmethyl)-maleimide. Eur J Biochem 101:395, 1979

What is claimed is:

1. A handheld detection device for detecting a biological marker comprising:
a reaction cell, said reaction cell comprising a combination of catalase, glucose, a biomolecular peroxide sensor, a capture antibody, an electrode, said electrode having immobilized on its surface the capture antibody and a recombinant fusion protein, said recombinant fusion protein having a redox activity and an immunoreactivity against the capture antibody; and
a potentiostat.

2. The detection device of claim 1, wherein the capture antibody specifically binds to an epitope in the biological marker.

3. The detection device of claim 2, wherein the capture antibody specifically binds to the epitope in the recombinant fusion protein.

4. The detection device of claim 1, wherein component of the recombinant fusion protein that provides the immunoreactivity against the capture antibody comprises SEQ ID NO:1.

5. The detection device of claim 4, wherein component of the recombinant fusion protein that provides the immunoreactivity against the capture antibody is located at the carboxy terminus of the recombinant fusion protein.

6. The detection device of claim 1, wherein the component of the recombinant fusion protein that provides the redox activity comprises SEQ ID NO:2.

7. The detection device of claim 1, wherein the recombinant fusion protein comprises SEQ ID NO:2 and SEQ ID NO:1.

8. The detection device of claim 7, wherein the recombinant fusion protein comprises SEQ ID NO:1 inserted within the amino acid sequence of SEQ ID NO:2.

9. The detection device of claim 7, wherein the recombinant fusion protein comprises SEQ ID NO:4.

10. The detection device of claim 7, wherein the recombinant fusion protein comprises SEQ ID NO:5.

11. The detection device of claim 1, wherein the capture antibody specifically binds a tumor marker.

12. The detection device of claim 11, wherein the tumor marker is selected from the group of PSA, HK2, TGFβ, her2, CA 15-3, CA-125, Cyfra 21-1, CEA, CD151, TPA, TPS, chromogrannin A, neuron specific enolase, β-HCG, α-fetoprotein, and LDH.

13. The detection device of claim 1, wherein the biomolecular peroxide sensor comprises a horseradish peroxidase.

14. The detection device of claim 1, wherein the capture antibody is biotinylated.

15. A disposable biosensor for screening for the presence of a biological marker in a sample comprising:
a reaction cell, said reaction cell comprising a combination of catalase, glucose, a biomolecular peroxide sensor, a capture antibody, an electrode, said electrode having immobilized on its surface the capture antibody and a recombinant fusion protein, said recombinant fusion protein having a redox activity and an immunoreactivity against the capture antibody.

16. A method of detecting a biological marker comprising:
obtaining a sample;
adding the sample to the detection device of claim 1;
applying an electrical signal to the detection device; and
measuring a magnitude of a current generated in the detection device, wherein the magnitude of the generated current is inversely proportional to the concentration of biological marker in the sample.

17. The method of claim 16, wherein the capture antibody specifically binds to an epitope in the biological marker.

18. The method of claim 17, wherein the capture antibody specifically binds to the epitope in the recombinant fusion protein.

19. The method of claim 16, wherein component of the recombinant fusion protein that provides the immunoreactivity against the capture antibody comprises SEQ ID NO:1.

20. The method of claim 19, wherein component of the recombinant fusion protein that provides the immunoreactivity against the capture antibody is located at the carboxy terminus of the recombinant fusion protein.

21. The method of claim 16, wherein the component of the recombinant fusion protein that provides the redox activity comprises SEQ ID NO:2.

22. The method of claim 16, wherein the recombinant fusion protein comprises SEQ ID NO:2 and SEQ ID NO:1.

23. The method of claim 22, wherein the recombinant fusion protein comprises SEQ ID NO:1 inserted within the amino acid sequence of SEQ ID NO:2.

24. The detection device of claim 16, wherein the capture antibody specifically binds a tumor marker.

25. The detection device of claim 24, wherein the tumor marker is selected from the group of PSA, HK2, TGFβ, her2, CA 15-3, CA-125, Cyfra 21-1, CEA, CD151, TPA, TPS, chromogrannin A, neuron specific enolase, β-HCG, α-fetoprotein, and LDH.

26. The method of claim 16, wherein the sample comprises whole blood, serum, plasma, urine, or saliva.

27. The method of claim 16, wherein the immunoreactivity is provided by a polypeptide comprising an epitope of a tumor marker.

28. The method of claim 16, wherein the recombinant fusion protein is prepared in yeast.

29. The method of claim 28, wherein the yeast is a methyltrophic yeast.

30. The method of claim 16, wherein the recombinant fusion protein is prepared by expressing a polynucleotide comprising both a glucose oxidase and the epitope, wherein the glucose oxidase and the epitope are operatively linked.

31. The method of claim 16, wherein the biomolecular peroxide sensor comprises a horseradish peroxidase.

32. The method of claim 16, wherein the capture antibody is biotinylated.

33. The method of claim 32, wherein the surface of the electrode further comprises avidin.

34. The method of claim 16, wherein the electrical signal comprises a voltage of about +50 mV.

35. The method of claim 16, wherein the measuring step comprises a potentiostat.

36. The method of claim 16, wherein the potentiostat is capable of measuring a current in the range of about 50 nanoampere to about 500 nanoampere.

37. A method of screening a patient for cancer comprising:
obtaining a sample from the patient;
adding the sample to a detection device, said detection device comprising a reaction cell, said reaction cell comprising a combination of catalase, glucose, a biomolecular peroxide sensor, a capture antibody, an electrode, said electrode having immobilized on its surface the capture antibody and a recombinant fusion protein, said recombinant fusion protein having a redox activity and an immunoreactivity against the capture antibody, and a potentiostat;
applying an electrical signal to the detection device; and
measuring a magnitude of a current generated in the detection device, wherein the magnitude of the generated current is inversely proportional to the concentration of a tumor marker in the sample;
and determining the presence of a cancer in the patient from the concentration of the tumor marker in the sample.

38. The method of claim 37, wherein the cancer is prostate cancer and the tumor marker is PSA, HK2, or TGFβ.

39. The method of claim 37, wherein the cancer is breast cancer and the tumor marker is HER2 or Cyfra 21-1.

40. The method of claim 37, wherein the cancer is ovarian cancer and the tumor marker is CA-125 or Cyfra 21-1.

41. The method of claim 37, wherein the cancer is colon cancer and the tumor marker is CEA.

42. The method of claim 37, wherein the cancer is lung cancer and the tumor marker is CD151, TPA, TPS, or Cyfra 21-1.

43. The method of claim 37, wherein the cancer comprises a neuro-endocrine tumor and the tumor marker is chromogrannin A, or neuron specific enolase.

44. The method of claim 37, wherein the cancer is testicular cancer and the tumor marker is β-HCG, alpha-feto protein, or LDH.

45. The method of claim 37, wherein the sample comprises whole blood, serum, plasma, urine, or saliva.

46. The method of claim 37, wherein the redox activity is provided by glucose oxidase.

47. The method of claim 37, wherein the immunoreactivity is provided by a polypeptide comprising an epitope of the tumor marker that binds specifically to the capture antibody.

48. The method of claim 37, wherein the electrical signal comprises a voltage of about +50 mV.

49. The method of claim 37, wherein the measuring step comprises a potentiostat

50. The method of claim 37, wherein the solution comprises about 1% glucose.

51. A kit for screening a patient comprising:
a handheld detection device for detecting a biological marker, said detection device comprising a reaction cell, said reaction cell comprising a combination of catalase, glucose, a biomolecular peroxide sensor, a capture antibody, an electrode, said electrode having immobilized on its surface the capture antibody and a recombinant fusion protein, said recombinant fusion protein having a redox activity and an immunoreactivity against the capture antibody.

52. A composition comprising SEQ ID NO:1.

53. A composition comprising a glucose oxide polypeptide and SEQ ID NO:1.

54. The composition of claim 53, wherein the composition is a recombinant fusion protein comprising SEQ ID NO:2.

55. The composition of claim 53, wherein the composition is a recombinant fusion protein comprising SEQ ID NO:4.

56. The composition of claim 53, wherein the composition is a recombinant fusion protein comprising SEQ ID NO:5.


SEQUENCE LISTING (5 sequences)

SEQ ID NO: 1 - protein, 7 aa, human
PEEFLTP

SEQ ID NO: 2 - protein, 587 aa, Penicillium amagasakiense
YLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSA

SEQ ID NO: 3 - protein, 604 aa, Aspergillus niger
MKTILSSSLVVSMAAACTLHRSSGIEASLLTDPKAVAGRTVDDIIAGGGLTGLTTAARLTENPNITVLVIESGFYESDRGPLVEDLNAYGEIFGSEVDHAYQTVELATNNLTELIRSGNGLGGSTLVNGGTWTRPHKVQVDSWETVFGNEGWNWENVAAYSLEAERARAPNAKQVAAGHYFDPSCHGTNGTVHVGPRDTGDDYTPIIDALMTTVENMGVPTKKDLGCGDPHGVSMFPNTLHEDQVRSDAAREWLLPNYQRPNLQVLTGQLVGKVLLDQNNTVPKAVGVEFGTHKANTFNVYAKHEVLLAAGSAVSPQILEHSGIGMKSILDTVGIDTVVDLPVGLNLQDQTIVLVSSRITSAGAGQGQVAIFATFNETFGDYAPQAHALLDAKLEQWAEEGVARGGFHNATALRIQYENYRDWLVNHNVAYSELFLDTAGAVSFTIWDLIPFTRGYVHITDADPYLRLVSYDPQYFLNELDLYGQAAASQLARNLSNTDAMQTYFAGETTPGDNPAYDASLSDWAEYIKYNFRPNYHGVGTCSMMKKELGGVVDSSARVYGVDSLRVIDGSIPPTQVSSHVMTVFYAMALKISAAILADYASSQ

SEQ ID NO: 4 - protein, 648 aa, penicillium amagasakiense
MVSVFLSTLLLSAAAVQAYLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSAAASGWGSIEPEEFLTPAAASFLEQKLISEEDLNSAVDHHHHHH

SEQ ID NO: 5 - protein, 722 aa, penicillium amagasakiense
MRFPSIFTAVLFAASSALAAPVNTTTEDETAQIPAEAVIGYSDLEGDFDVAVLPFSNSTNNGLLFINTTIASIAAKEEGVSLEKREAEASASGWGSIEPEEFLTPLQYLPAQQIDVQSSLLSDPSKVAGKTYDYIIAGGGLTGLTVAAKLTENPKIKVLVIEKGFYESNDGAIIEDPNAYGQIFGTTVDQNYLTVPLINNRTNNIKAGKGLGGSTLINGDSWTRPDKVQIDSWEKVFGMEGWNWDNMFEYMKKAEAARTPTAAQLAAGHSFNATCHGTNGTVQSGARDNGQPWSPIMKALMNTVSALGVPVQQDFLCGHPRGVSMIMNNLDENQVRVDAARAWLLPNYQRSNLEILTGQMVGKVLFKQTASGPQAVGVNFGTNKAVNFDVFAKHEVLLAAGSAISPLILEYSGIGLKSVLDQANVTQLLDLPVGINMQDQTTTTVSSRASSAGAGQGQAVFFANFTETFGDYAPQARDLLNTKLDQWAEETVARGGFHNVTALKVQYENYRNWLLDEDVAFAELFMDTEGKINFDLWDLIPFTRGSVHILSSDPYLWQFANDPKFFLNEFDLLGQAAASKLARDLTSQGAMKEYFAGETLPGYNLVQNATLSQWSDYVLQNFRPNWHAVSSCSMMSRELGGVVDATAKVYGTQGLRVIDGSIPPTQVSSHVMTIFYGMALKVADAILDDYAKSAAAAASFLEQKLISEEDLNSAVDHHHHHH